(19) United States
(12) Patent Application Publication
(10) Pub. No.: US 2021/0283260 A1
(43) Pub. Date: Sep. 16, 2021

(54) A CELLULAR SOLID MATERIAL DRUG CARRIER COMPRISING CELLULOSE NANOFIBERS (CNF) WHEREIN THE CELLULAR SOLID MATERIAL COMPRISES CLOSED CELLS

(71) Applicants: Cellutech AB, Stockholm (SE); University of Copenhagen, Copenhagen K (DK)

(72) Inventors: Anna Justina Hanner, Malmo (SE); Korbinian Löbmann, Copenhagen N (DK); Anette Müllertz, Charlottenlund (DK); Daniel Bar-Shalom, Kokkedal (DK); Lars-Erik Wågberg, Stockholm (SE)

(21) Appl. No.: 16/316,192

(22) PCT Filed: Jul. 7, 2017
(86) PCT No.: PCT/SE2017/050765

§ 371 Date: Jan. 8, 2019

(30) Foreign Application Priority Data
Jul. 8, 2016 (SE) 1651018-2

(51) Int. Cl. A61K 47/38; A61K 9/00; A61K 9/12; A61K 9/14; B01J 20/28; D21H 11/18; D21H 19/34

(52) U.S. Cl. CPC A61K 47/38; A61K 9/0065; A61K 9/006; A61K 9/122; D21H 19/34; A61K 9/146; A61K 9/148; B01J 20/28045; D21H 11/18; A61K 9/145

(57) ABSTRACT

The present invention relates to a structure for the controlled release of at least one active substance, where the structure comprises the active substance and a cellular solid material comprising cellulose nanofibers (CNF). The structure has a density of less than 1000 kg/m3, and the cellular solid material comprises closed cells. The invention further relates to a method for preparing the structure; as well as the use of the structure.

### FIELD OF THE INVENTION

The present invention relates to a structure for the controlled release of an active substance, where the structure comprises the active substance and a cellular solid material comprising cellulose nanofibers (CNF) or modified CNF; a method for preparing the structure; and the use of the structure for controlled release of said active substance.

### TECHNICAL BACKGROUND

Controlled release of active substances adjusts the release of said substances to accommodate to a desired effect, in timing or in space or both. The concept is applicable in different fields, such as in medicine, agriculture, industrial processes, personal care, household products, nutrition and food, dietary supplements, veterinary products and other applications where controlled release of an active substance is desired. In the case of drugs, controlled release is used to alter the pharmacokinetics of the drug. By controlling the release of a pharmaceutically active substance, patient compliance and safety can be improved as a predictable drug release or a lower frequency of administration may be obtained. Controlled-release is in particular important for drugs with short biological half-lives, in that it may improve the bioavailability of the drug. Controlled drug delivery prolongs action and also attempts to maintain drug levels within the therapeutic window and enables optimal drug concentrations in the blood as a function of time and as a consequence fewer side-effects are expected such as drug toxicity and less drug waste.

Cellulose and its derivatives are widely used as pharmaceutical excipients. Among different celluloses the microcrystalline cellulose (MCC), carboxymethyl cellulose and others are commonly used in solid dose forms, such as in tablets, as fillers and binders, and crosslinked sodium carboxymethyl cellulose (croscarmellose sodium) is commonly used as a disintegrant in pharmaceutical manufacturing. Ethyl cellulose is used in pharmaceutical industry as a coating agent, flavouring fixative, tablet binder and filler, film-former, and also in modified release dosage forms. Hydroxypropylmethyl cellulose (HPMC), also known as hypromellose, has also been used as a rate-controlling polymer for sustained-release dose forms.

Cellulose is the most abundant renewable natural polymer on earth and is used in large volumes on an industrial scale. Cellulose chains with β-(1-4)-D-glucopyranose repeating units are packed into long nanofibrils in the plant, with cross-sectional dimension of 5-30 nm depending on the plant source. The parallel organization of the cellulose chains, held together by hydrogen bonds and organized in sheets, gives a crystal structure with a Young's modulus of approximately 130 GPa. These crystal domains are the reason why native cellulose, crystal form I, has such a high modulus and strength. Nanofibrils from cellulose (CNF) have opened a new field as construction units for nanoscale materials engineering. These entities can be released from the pulp fiber cell wall by mechanical disintegration (A. F. Turbak, et al., J Appl Polym Sci, 1983, 37, 815), which is facilitated by an enzymatic or chemical pre-treatment of the pulp fibers (M. Henriksson, et al., Eur Polym J, 2007, 43, 3434; T. Saito et al., Biomacromolecules, 2007, 8, 2485; and M. Ghandapour, Biomacromolecules, 2015, 16, 3399-3410).

Drug delivery structures based on cellulose nanofibers (CNF) is a novel concept that has been studied (Kolakovic, et al., International Journal of Pharmaceutics 2012, 430, 47-55; Kolakovic, et al, Eur. J. Pharm. Biopharm. 2012, 82, 308-315; Gao, et al., ChemPlusChem 2014, 79, 725-731). Kolakovic et al. present drug-loaded CNF microparticles and CNF films, see also WO2013/072563. Valo et al., Eur. J. Pharm. Sci. 2013, 50, 69-77 prepared freeze-dried CNF aerogels containing drug nanoparticles for drug release.

Cervin et al. (Biomacromolecules, 2013, 14, 503-511) demonstrated the use of CNF for Pickering stabilization in foams in combination with a surfactant. WO2014/011112A1 discloses the preparation of hydrophobized wet foams from anionic CNF hydrophobized by adsorption of cationic hydrophobic amines. WO2016/068771 and WO2016/068787 present cellular solid materials comprising cellulose nanofibers (CNF) and an anionic surfactant or a non-ionic surfactant and their preparation.

### SUMMARY OF THE INVENTION

The objective of this invention is to provide a structure for controlled release of at least one active substance.

One aspect of the present invention is a structure for the controlled release of at least one active substance, wherein the structure comprises said active substance and a cellular solid material comprising cellulose nanofibers (CNF), wherein the structure has a density of less than 1000 kg/m³ and more than 10% of the total volume of the cells of the cellular solid material are closed cells.

Another aspect of the invention is a method for preparing a structure for controlled release of at least one active substance, wherein the structure comprises said active substance and a cellular solid material comprising cellulose nanofibers (CNF), the method comprising:
a) providing a dispersion comprising CNF in an aqueous solvent,
b) adding at least one active substance to the dispersion in (a) to obtain a mixture;
c) preparing a wet foam from the mixture obtained in (b), wherein the wet foam has a density less than 98% of the density of the mixture prior to foaming; and
d) drying the wet foam obtained in (c) to obtain a structure comprising a cellular solid material and at least one active substance.

A further aspect of the present invention is the use of a cellular solid material comprising cellulose nanofibers (CNF) and at least one active substance in a structure for controlled release of said active substance.

An additional aspect of the present invention is the use of a structure according to the present invention.

### BRIEF DESCRIPTION OF THE DRAWINGS

FIG. 1 schematically illustrates a method for forming a cellular solid material (7).

FIG. 2 illustrates a layered composition (4) prepared from a combination of layers of a cellular solid material (1); a structure according to the invention, i.e. a cellular solid material comprising an active substance (2); and a wet foam (3) that upon drying will glue together the cellular solid material layers (1).

FIG. 3 shows cross-sections of the resulting cellular solid materials loaded with 21 wt % (FIG. 3a), 50 wt % (FIG. 3b) furosemide, and a close-up of the cell wall of the different cellular solid materials (FIG. 3c and FIG. 3d), respectively, containing undissolved furosemide particles.

FIG. 4 presents FTIR spectra for a neat CNF film, the active substance furosemide and cellular solid materials with 21 wt % and 50 wt % furosemide.

FIG. 5 shows the cumulative drug release as a function of time for furosemide samples (a tablet and cellular solid materials loaded with furosemide (21 wt % and 50 wt %)).

FIG. 6 illustrates different thicknesses and shapes of cellular solid materials, as well as an example of loading a capsule with a cellular solid material (7).

FIG. 7 shows cross-sections of neat CNF/lauric acid cellular solid material (a); a film loaded with riboflavin 14 wt % (b); a cellular solid material loaded with riboflavin 14 wt % (c), and 50 wt % (d), respectively; a close-up of the cell wall with a riboflavin crystal (e); and a close-up of the cell wall neat CNF/lauric acid cellular solid material (f).

FIG. 8 presents FTIR spectra for a neat CNF film, the active substance riboflavin, and cellular solid materials with 14 wt % and 50 wt % riboflavin, respectively.

FIG. 9 presents the IR-spectra of the active substance indomethacin in its pure crystalline (γ-form and α-form) and amorphous indomethacin (INDam), a neat nanocellulose film (CNF) and nanocellulose films loaded with 21 wt % (21% IND) and 51 wt % indomethacin (51% IND), respectively. The IR-spectra for a cellular solid material with 21 wt % indomethacin overlapped with the IR-spectra for the film with 21 wt % indomethacin and therefore only one of these IR-spectra is included.

FIG. 10 presents the cumulative drug release as a function of time for structures containing in (a) riboflavin as active substance in a tablet (Tablet), a film (Film), thin cellular solid materials comprising 14 wt % (14% Ribo) and 50 wt % riboflavin (50% Ribo), respectively, and a thick cellular solid material comprising 14 wt % riboflavin (Thick cellular solid, 14%), and in (b) a film (Film), and cellular solid materials of two different thicknesses (Thin cellular solid, 14% Ribo, and Thick cellular solid, 14%), all comprising 14 wt % riboflavin.

FIG. 11 presents release of indomethacin from different structures: in figure (a) the cumulative drug release of indomethacin as a function of time for a film comprising 21% IND (21% IND), a cellular solid material comprising 21% IND (Cellular solid) and a film comprising 51% IND (51% IND); and in figure (b) the intrinsic dissolution of indomethacin (mg cm⁻²) as a function of time for a film comprising 21% IND (21% IND), a cellular solid material comprising 21% IND (Cellular solid), a film comprising 51% IND (51% IND), IND amorphous (INDamorph) and in crystalline form (α-form).

FIG. 12 presents the total amount of riboflavin (mg) that has passed a film as a function of time (min). The solid line is a best fit to the experimental data.

FIG. 13 presents the total amount of riboflavin (mg) that has passed a cellular solid material as a function of time (min). The solid line is a best fit to the experimental data.

### DETAILED DESCRIPTION OF THE INVENTION

All words and abbreviations used in the present application shall be construed as having the meaning usually given to them in the relevant art, unless otherwise indicated. For clarity, some terms are however specifically defined below. It should be noted that embodiments, features, or advantages described in the context of one of the aspects and/or embodiments of the present invention may also apply mutatis mutandis to all the other aspects and/or embodiments of the invention.

The term “CNF” is used herein for cellulose nanofibers liberated from wood pulp or from other sources, for example selected from the group consisting of plants, tunicate, and bacteria by means of mechanical disintegration, often preceded by a chemical pretreatment, such as by oxidation with 2,2,6,6-tetramethylpiperidine-1-oxyl (TEMPO) giving TEMPO-oxidized CNF, or by carboxymethylation giving carboxymethylated CNF; or by enzyme-treatment, such as by endoglucanases, giving enzymatic CNF. CNF typically have a smallest dimension in the range 2-100 nm, while the length can be several micrometers, such as up to 10 μm, and therefore the aspect ratio of CNF (ratio of length to diameter) is very large. An advantage of using CNF from wood-pulp is the abundance of wood-based cellulose and the existing, efficient infrastructure for the handling and processing of pulp and fibers.

Throughout the present description the term “cellular solid material” is used for an assembly of cells packed together, and where the cell wall is of a solid material. The cell wall may comprise both the edges and faces of the cell. If the solid material is contained in both the edges and faces of the cell, so that the cell is sealed off from its neighbours, the cells of the cellular solid material are closed-cells. If the cell wall, i.e. the solid material, is contained in the edges only, so that the cells connect to their neighbours through open faces, the cells of the material are open-cells.

The term “excipient” is used herein for a natural or synthetic substance formulated alongside the active substance, such as for the purpose of stabilization; to bulk up the formulation containing the active substance, e.g. bulking agents, fillers, diluents; or to confer a therapeutic enhancement on the active ingredient in the final dosage form, such as facilitating drug absorption, reducing viscosity, control the release, or enhancing solubility. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active substance and other factors.

The term “controlled release” as used herein, intends to encompass delivery of an active substance in response to stimuli or time. Examples of such stimuli are the use of enzymes, pH, light, temperature, osmosis, moisture, ultrasonic, force, pressure, and erosion. Controlled release of an active substance is usually understood to denote a release profile that extends the release to be slower than the immediate release of the active substance from a conventional dosage form, but it may also include enhancing the release to make the active substance reach the target site even faster than for the conventional dosage form. The term encompasses enhanced or fast release, pulsed release, sustained release, extended release and prolonged release; as well as delayed release. The controlled release of an active substance may not only prolong the action of the substance but may also maintain the levels of the active substance within the effective window to avoid peaks in the concentration of the substance that may potentially be harmful, and to maximize efficiency of the substance.

Throughout the present description the term “sustained release” is used for a dosage form that shows slower release of the active substance(s) than that of a conventional release dosage form administered by the same route. A sustained release formulation of a drug may maintain the drug concentration within the therapeutic window for a prolonged time, which allows a reduction in frequency of the drug administration in comparison with conventional dosage forms. “Delayed release” is used herein for formulations that delay the release of the active substance until the formulation has reached its target site or at a particular time. The term “fast release” or “burst release” is used herein for formulations that enables a quick release of the active substance after administration, for example by uptake through the mouth palate or gums following oral administration. Combinations of the above are also contemplated such as delayed burst release. The term “enhanced release” is used herein for formulations that enables a more complete or faster release of the active substance, such as all or most of the active substance included in the dosage form, compared with the conventional dosage form.

The structure according to the present invention may be used in several areas, for example in pharmaceuticals, such as for release of pharmaceutically acceptable agents, as well as in medical devices; industrial applications, such as in fermentation, release of catalysts, release of coolants, or in chemical reactions, such as for release of chemical reagents; food science applications, such as transport and release of ingredients of functional food; household applications, such as in disinfectants, dish soap, dish washing tablets, detergents, and air-fresheners; personal care, such as cosmetics, and perfumes; veterinary medicine; and agriculture, such as for release of fertilizers, pesticides, and micronutrients. An active substance used in a structure according to the present invention, is thus a substance that should be transported and delivered from the structure at a specific target, or at a controlled rate, or both, to achieve or promote a desired effect.

The active substance may be selected from small-molecules, such as molecules with a molecular weight of less than 900 daltons; macromolecules, such as molecules with a molecular weight of 900 daltons or more; biopharmaceutical drugs; or a vehicle, such as for a vaccine and nonspecific immune response enhancers. The active substance should be able to diffuse through the cellular solid material following the exposure of the structure to a releasing agent, such as, but not limited to, a solvent, a body fluid and a tissue. Examples of active substances for use in the present invention are selected from pharmaceutically acceptable agents, catalysts, chemical reagents, nutrients, food ingredients, enzymes, bactericides, pesticides, fungicides, disinfectants, fragrances, flavours, fertilizers, and micronutrients. Preferably the active substance is a pharmaceutically acceptable agent. The pharmaceutically acceptable agent may be a therapeutically, prophylactically and diagnostically active substance.

The relative amount of the active substance depends on the intended use of the structure for controlled release. The structure according to the present invention may comprise up to and including 90 wt %, up to and including 80 wt %, or up to and including 50 wt %, of an active substance, as calculated on the total weight of the structure. The structure according to the present invention may comprise at least 0.2 wt %, or at least 0.5 wt % active substance, calculated on the total weight of the structure.

The cellular solid material used in the present invention may be used as an excipient or as a coating for the active substance. The structure according to the present invention may, however, also contain further excipients in addition to the cellular solid material.

The CNF used in the cellular solid material and in the method for its manufacturing according to the present invention may be cellulose nanofibers selected from the group consisting of enzymatic CNF, TEMPO-CNF, phosphate functionalized CNF, glycidyltrimethylammonium chloride functionalized CNF, and carboxymethylated CNF, or a combination of two or more of these CNFs. These CNFs might be further chemically modified in a pre-treatment before preparation of the structure according to the invention or as a post-treatment. The CNF used in the cellular solid material according to the present invention may be anionic, cationic or non-ionic.

The structure according to the present invention may comprise at least 10 wt %, at least 20 wt %, at least 30 wt %, at least 40 wt %, at least 50 wt %, or at least 60 wt % CNF, calculated on the total weight of the structure. The structure may comprise up to and including 99.8 wt % CNF, up to and including 99.5 wt % CNF, up to and including 99 wt %, up to and including 95 wt %, up to and including 90 wt %, up to and including 80 wt %, or up to and including 70 wt % CNF, calculated on the total weight of the structure.

The present invention thus concerns a structure for the controlled release of active substances, wherein the structure comprises at least one active substance and a cellular solid material consisting of cellulose nanofibers (CNF), or modified CNF, wherein the structure has a density of less than 1000 kg/m³. In a preferred embodiment, the structure according to the present invention may have a density of less than 500 kg/m³, or less than 100 kg/m³, or less than 50 kg/m³. The density of the cellular solid material may be at least 1 kg/m³, or at least 5 kg/m³. A low density structure can float in aqueous medium, such as in the gastric fluids.

The structure according to the present invention may be a floating drug delivery structure (FDDS). The structure may also be a part of a floating drug delivery structure. An advantage with a floating drug delivery structure is that it may control the release of the active substance to occur at the target site, for example at a specific site in the gastrointestinal tract, and also the rate of the release of the active substance at that target site. Typically, the gastric retention time of a substance is in average 1.5 hours and very variable and unpredictable. A structure for delayed release, i.e. gastro retentive structures, can improve bioavailability of drugs.

The porosity of the cellular solid material represents the total volume of cells present in the cellular solid material, i.e. both closed and open cells. The porosity, ϕ), of a cellular solid material is calculated by using equation [1], where ρ is the density of the cellular solid material according to the present invention and ρ_{cell wall} is the density of the solid dry cell wall. For a cell wall consisting of dry solid cellulose the density is 1.5 g/cm³.

$$ϕ = 1 - \frac{ρ}{{ρ}_{cell wall}} \qquad [ 1 ]$$

The porosity of the cellular solid material used in the structure according to the present invention may be at least 67%, or at least 93%, or at least 97%.

The theoretical density of the cell wall (ρ_{cell wall}) is calculated by equation [2]:

ρ_{cell wall}=ν_{VCF}ρ_{CNF}+ν_{active sub}ρ_{active sub} [2]

where ν_{CNF} is the volume fraction of the CNF and ν_{active sub} (=1−μ_{CNF}) is the volume fraction of the active substance. ρ_{CNF} is the density of dry solid CNF and ρ_{active sub} is the density of the active substance.

The proportion of closed cells in relation to the total volume of cells of the cellular solid material can be expressed as a volume percentage (% V_{C}) and is calculated by using the equation [3], where:

$$\% {V}_{c} = \frac{( \frac{{m}_{cell wall} + {m}_{x}}{{ρ}_{w}} ) \cdot ( \frac{{m}_{cell wall}}{{ρ}_{cell wall}} )}{{V}_{CSM} \cdot ϕ} \cdot 100 \qquad [ 3 ]$$

mₓ is the mass of the extra weight that is needed to add to a piece of cellular solid material of known volume (V_{CSM}), so that the piece of cellular solid material (which originally floats due to the existence of closed cells) is immersed in water and hold under the surface of water,
m_{cell wall} is the mass of the cell wall for the dry piece of cellular solid material of known volume V_{CSM},
ρ_{w} is the density of water,
ρ_{cell wall} is the density of the solid dry cell wall,
ϕ is the porosity of the cellular solid material as calculated with equation [1].

The measurements should preferably be made on a piece of cellular solid material with the dimensions 5*5*2 cm (L*B*H), thus providing a known volume, V_{CSM}, of 50 cm³.

A cellular solid material comprising closed cells provides for a sustained release of the active substance from the structure compared to a structure wherein the cells are open cells or a structure in the form of a film. Preferably more than 10% of the total volume of the cells of the cellular solid material in the structure according to the present invention are closed cells. More preferably, more than 30%, more than 50%, or more than 90%, of the total volume of the cells are closed cells. The diameter or the largest cross section of the cells may be at least 10 μm, at least 200 μm, or at least 300 μm. The diameter or the largest cross section of the cells may be as high as 10000 μm, or 5000 μm, or 1000 μm, or 800 μm.

The structure of an excipient or coating impacts the release profile of the enclosed active substance. In the present invention, the cellular solid material contains impermeable objects in the form of gas-bubbles trapped in the closed cells. The release of an active compound from such cellular solid materials will be typically diffusion-controlled, since the gas-bubbles provide for a longer and tortuous path for the active substance that is diffusing through the cellular material surrounding the bubbles. The diffusion through such a material will therefore be slower compared to a similarly composed CNF film of comparable thickness that does not comprise a cellular solid material. Unmodified CNF based films have excellent barrier properties in the dry state, but these properties are quickly lost in the wet state due to the disruption of the strong hydrogen bonds between nanofibers which are mainly responsible for the high barrier properties in the dry state causing an enclosed substance to be rapidly released. An advantage with the cellular solid material used in the present invention is that the cellular structure and gas bubbles may be preserved during dissolution. The ability of CNF to be wetted and still have the cellular structure and gas bubbles preserved provides for modified diffusion of the active substance through the CNF material compared to the diffusion through a film. Adsorption, diffusion and release kinetics of the active substance in a cellular solid material of CNF in the wet state may thus be controlled. Further, the preserved cells and high porosity may provide the material with buoyance power.

From a pharmaceutical perspective, tailoring of the dissolution characteristics of a drug can be of immense importance, as it can improve the bioavailability and/or pharmacokinetics of a drug. During the gastro intestinal transit time, the drug needs to dissolve sufficiently in order to be absorbed by the body and to have a satisfactory therapeutic effect. For poorly soluble substances, this can often only be achieved by solubility- or dissolution-enabling drug delivery strategies, such as preparing the amorphous form of the substance. However, many amorphous substances re-crystallize upon storage. An advantage with the structure comprising a cellular solid material comprising CNF as in the present invention is that the amorphous form of the active substance may be maintained without re-crystallizing when stored. The solid state of the drug within the cellular solid material comprising CNF may range from crystalline (different polymorphs, solvates, hydrates, co-crystals and salts), liquid crystalline to the amorphous form, or a combination of the different solid forms.

A prolonged release at the absorption site may enable a higher bioavailability of poorly soluble substances. Slow release profiles may be also important for poorly soluble drugs with a narrow therapeutic window where fast release formulations could otherwise result in adverse effects. The therapeutic window is the concentration range between the therapeutically effective dose and a dose that results in intolerable side or toxic effects. In order to avoid the undesirable effects, such drugs are often given in low doses several times a day. Using a slow release formulation would allow a therapeutic effect over several hours up to the gastro intestinal transit time of the formulations. Fast release formulations, on the other hand, may be desirable in many other cases to ensure an immediate drug action after administration, for example for treating an ongoing myocardial infarction or an epileptic seizure.

An advantage with using a cellular solid material comprising cellulose nanofibers (CNF) in the structure for controlled release of an active substance according to the present invention is that the structure may be made using conventional industrial paper conveyer structures. A solid dosage form could easily be individualized by cutting out appropriately sized pieces of the cellular solid material containing the desired amount of the active substance. Personalized doses are of great interest in pharmaceutical industry but also for better drug delivery to the patient.

The structure comprising cellular solid material of cellulose nanofibers (CNF) and at least one active substance according to the present invention may be used in a layered assembly, such as an envelope, for release of an active substance, a particle, multiple particles, or a liquid. For example, such assemblies may comprise one or more layers of a cellular solid material coating a structure comprising cellular solid material of cellulose nanofibers (CNF) and at least one active substance, a ravioli configuration being a suitable analogy. An embodiment of the present invention where a cellular solid material of cellulose nanofibers (CNF) is used in a layered assembly (4), such as an envelope, is illustrated in FIG. 2, where outer layers of a solid cellular material (1) cover a middle layer comprising a piece of a solid cellular material comprising at least one active substance (2) and a wet foam (3) which after drying will glue the solid cellular materials (1) together. Using different combinations of layers of cellular solid material and active substances in layered assemblies enables further tailoring of the controlled release of the active substances.

Using a cellular solid material comprising CNF in a structure according to the present invention, may provide for a slower and better controlled release of the active substance compared to films comprising CNF and the corresponding active substance. Increased thickness may prolong the release without increasing the weight of the material compared to a flat film. In a cellular solid material, the active substance diffuses through the CNF based cell walls in the material, which efficiently slows down the release rate. The presence of closed cells, such as intact gas-bubbles, may create a tortuous and extended diffusion path as the drug cannot diffuse through the intact gas-bubbles, only the cell-wall, which reduces the apparent diffusion of the active substance. A structure comprising an active substance on or near the outer surface of the cellular solid material may provide for an initial immediate release of said active substance, which can be followed by a slower release of active substance, which may be the same substance or a different substance, located inside the cellular solid material. Further, the presence of CNF may increase the solubility of the active substance, for example indomethacin.

Controlled release may for example be used in pharmaceutical devices and compositions; cosmetics; personal care; household applications; food science applications; veterinary medicine; and agriculture. The purpose of pharmaceutical devices and compositions concentrate on release of the pharmaceutically active substance. Cosmetics, personal care and food science applications often centre on odour or flavour release. In the structure according to the present invention the controlled release may be a delayed release, a sustained release, a fast release, or a burst release. A fast release may be provided by puncturing the cells in the cellular solid material. Such puncturing may for example be made by chewing the structure according to the invention for obtaining fast release of an enclosed active substance in the oral cavity. Preferably, the controlled release from the structure according to the present invention is a delayed release or sustained release, more preferably a sustained release. The structure according to the present invention could be used for gastro-retentive drug delivery with prolonged drug delivery at the absorption site, i.e. the stomach and the upper intestine.

Structures for controlled release may be used in oral applications, such as modified and prolonged release dosage forms, gastro retentive drug delivery, drug delivery from chewing cellular solid materials where the cellular solid materials remains stable during chewing, drug delivery from chewing cellular solid materials where the cellular solid materials collapses during chewing, bioadhesive delivery, e.g. adhesive films/cellular solid materials with continuous drug release to the intestine, chewing substitute for chewing gums, and sandwich cellular solid materials; topical applications, such as sublingual applications for fast release medications; transdermal applications, for example long acting mosquito repellent products, or active plasters; and buccal applications, such as in bioadhesive (buccal) delivery for prolonged release in for example maintenance treatments (e.g. baseline nicotine, anti-inflammatory drugs, pain killers, drugs with extensive first pass metabolism, drugs with narrow window of absorption) and saliva stimulating, lubricant releasing cellular solid materials; continuous antibiotics release in surgery; colonic delivery by making it degradable by microorganisms; vaginal applications; rectal applications; and nasal applications.

Examples of specific applications of the present invention for fast release formulations are sublingual application for fast release medication, such as for treating migraine; a heart medicine, e.g. release of nitroglycerine; a protein; vaccine; anticonvulsant, anticancer treatment; rescue medicine, such as for treating epilepsy, pain, or Parkinson; or for fast release of nicotine to obtain a kick.

Structures for controlled release according to the present invention may also find use in paediatrics, as easy to swallow cellular solid materials. The cellular solid material lubricates upon contact with saliva and makes drug delivery easier to patients that have problems to swallow tablets. The structure may also be cut in smaller units that will be easier to swallow. The structure may also be provided as edible sachets.

The structures for controlled release according to the present invention may also be used for dressing, i.e. wound bandage, such as in carrier material for wounds, chronic wounds, or burnings; in plaster material; intra-wound coagulation promoter; and for antibiotics release. Another application for structures according to the present invention is use in personalized medicine. The structure may be produced on conveyer belts and then cut into custom sized pieces containing the desired amount of the active substance. A further application for the structures according to the present invention is in taste masking, which is useful in for example paediatrics, and veterinary medicine: In such applications encapsulation of well tasting substances within the cellular solid material may mask the taste of other substances.

Other examples of applications of the structures according to the present invention are in tissue engineering; perfumes, such as for long acting perfume carrier for perfume samples, or room refreshments; filter material, such as for purification of nanoparticles by filtration, or in a molecular filter; disinfectants; and antifungal in aquarium and aquaculture.

The structure according to the present invention may be used for administration of a pharmaceutically active substance, wherein the administration of the pharmaceutically active substance, is selected from any one of oral; topical, including the buccal mucosa; transdermal; subdermal; intracavitary, for example administration in the uterus, peritoneum, pleura or bladder, preferably administration in uterus, or bladder; rectal; vaginal; and intranasal administration, or a combination of two or more of these. Preferably, the administration is selected from any one of oral; topical; transdermal; subdermal; intracavitary; and intranasal administration, or a combination of two or more of these. The structure according to the present invention may be a buccal mucosa drug delivery structure.

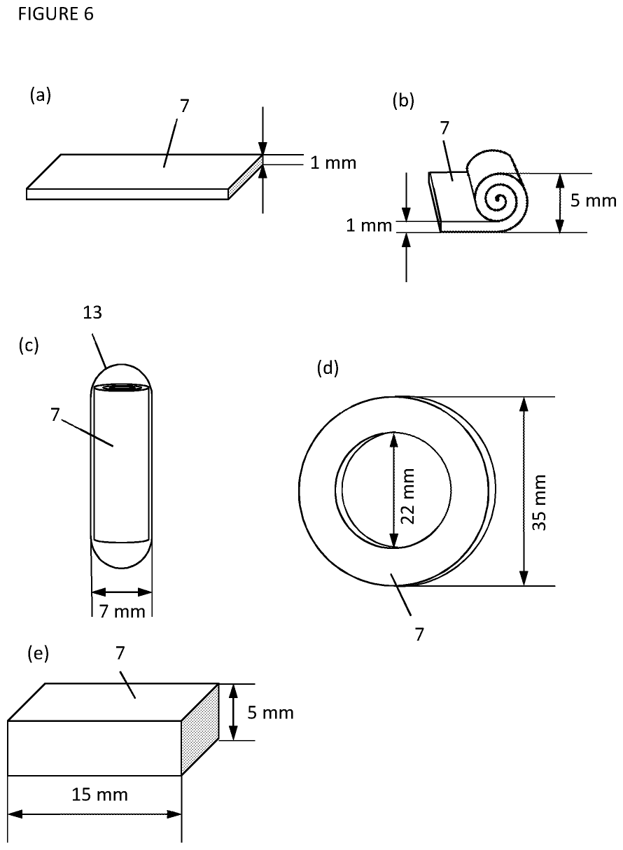

The shape of the dosage form may affect the controlled release, for example the gastric residence time of floating devices. FIG. 6 is illustrating the versatility of a structure according to the present invention. Cellular solid materials of different thicknesses (7) (FIGS. 6a and 6e); shapes, such as rings or slabs (FIGS. 6a, 6d and 6e); and drug loading may be prepared. The flexibility of thin cellular solid materials (7) (FIG. 6a) allows a structure according to the present invention to be folded or rolled (FIG. 6b) into a smaller object, which may be delivered in a capsule suitable for swallowing (13) (FIG. 6c). The structure for controlled release of at least one active substance according to the present invention may be provided in different configurations, such as a tablet; a pill; a lozenge; a capsule; a granule; a sachet; a chewing gum; a layered structure, such as a sandwich laminate; an injectable carrier; a gel; a lotion; transdermal patches; a bioadhesive; a scaffold, such as a carrier for long acting perfume samples or room refreshments; an implant; and other devices, such as filters. Preferred configurations for controlled release of a pharmaceutically acceptable agent are selected from a tablet; a pill; a lozenge; a capsule; a granule; a sachet; a chewing gum; a layered structure; an injectable drug carrier; a gel; transdermal patches; a bioadhesive; a scaffold; a device, such as a vaginal ring; and an implant, such as an implant for temporary release or non-temporary release in or on the body, for example a contraceptive implant. The final product may also be presented as pieces cut from sheet cellular solid material or extruded profiles or directly moulded into forms. Further, the structure according to the present invention may be provided with a coating. A coating may mask the taste of the structure, contain a loading dose, i.e. a discrete amount of active substance to be released without delay after administration, further modify the release profile, protect the structure, limit the exposed surface where the active substance can exit the structure, improve the organoleptics, such as the texture or the feel of the structure in the mouth.

The present invention further relates to a method for preparing a structure for controlled release of at least one active substance comprising a cellular solid material comprising cellulose nanofibers (CNF) and at least one active substance, comprising:
a) providing a dispersion comprising cellulose nanofibers (CNF) in an aqueous solvent,
b) adding at least one active substance to the dispersion in (a) to obtain a mixture;
c) preparing a wet foam from the mixture obtained in (b), wherein the wet foam has a density less than 98% of the density of the mixture prior to foaming; and
d) drying the wet foam obtained in (c) to obtain a structure comprising a cellular solid material and at least one active substance.

The CNF concentration in the dispersion in step (a) may be at least 0.0001 wt %, at least 0.2 wt %, at least 0.3 wt %, at least 0.4 wt %, or at least 0.5 wt %, calculated on the total weight of said dispersion. Dispersions of at least 1 wt % CNF, calculated on the total weight of the dispersion, may also be used in the method according to the present invention. An advantage with higher concentrations of CNF is that the time for drying the wet foam is decreased. The viscosity of CNF dispersions increases substantially when the CNF concentration is increased, the upper limit for the concentration of CNF will depend on the available foaming setup, e.g. the capacity of the mixer. Typically, the concentration of CNF in the dispersion in step (a) may be up to and including 30 wt %, or up to and including 10 wt % CNF, or up to and including 2 wt % CNF or up to and including 1 wt %, calculated on the total weight of said dispersion.

The aqueous solvent used for making the CNF dispersion in step (a) may be water, or a mixture of water and an organic solvent, such as ethanol. Such mixture of water and an organic solvent may have a water content of at least 0.1%, at least 3%, at least 10%, at least 50%, at least 70%, at least 90%, or at least 95%, calculated on the total weight of the aqueous solvent.

It is possible to add one or more surfactants in step (a) or (b), such as anionic, cationic or non-ionic surfactants, in addition to the active substance. However, and advantage with the structure for controlled release according to the present invention is that it may be prepared without addition of any surfactant, while still providing a cellular solid material. Gas-bubbles formed during foaming in step (c) are stabilized and preserved because of the presence of active substance and the inherent physicochemical properties of CNF. In certain embodiments, it may be advantageous to minimize the number of ingredients and use only a few or only well-characterized ingredients, for example in a method for preparing a structure that will be subject to regulatory registration, such as a pharmaceutical composition. The present method thus benefits from that the same active substance may first be used for stabilizing the bubbles in the solid cellular material that is part of the structure according to the invention, and that the same active substance later can be released from the resulting structure under controlled forms. Although the method according to the present invention does not require the addition of further components, such as plasticizers, crosslinking agents, inorganic or organic nanoparticles, clay, cellulose, nanocrystals, or polymers; such components may still be added in a method for preparation of a structure to provide it with certain properties that are required for the intended use, for example in industrial applications.

The active substance added in step (b) may either be poorly soluble in aqueous media, such as indomethacin, furosemide and lauric acid sodium salt, or water-soluble. The active substance added in step (b) may be selected from pharmaceutically acceptable agents, catalysts, chemical reagents, nutrients, food ingredients, enzymes, bactericides, pesticides, fungicides, disinfectants, fragrances, flavours, fertilizers, and micronutrients. Preferably, the active substance added in step (b) is a pharmaceutically acceptable agent. More than one active substance may be added in step (b).

One or more excipients, such as pharmaceutically acceptable excipients, may also be added in step (b), along with the addition of the active substance. The density of the mixture obtained in (b) is determined by dividing the weight of the components in the mixture with the volume of the mixture.

The preparation of a wet foam in step (c) of the method may be performed by introducing a gas into the mixture obtained in step (b). The gas may be introduced by mixing; such as beating, agitation, shaking, and whipping; bubbling or any other means suitable for formation of foam. Thus, foaming may be performed by mixing the mixture comprising CNF and at least one active substance in the presence of a gas. Alternatively, foaming may be performed by blowing a gas or adding a foaming agent into the mixture. The density of the wet foam prepared in step (c) may be determined by dividing the weight of the components in the mixture prior to foaming with the volume of the wet foam. It is possible to add additional active substances, such as riboflavin, and/or one or more excipients to the wet foam prepared in step (c). The wet foam obtained in (c) of the present method is stable for a period long enough to allow it to be dried without collapsing and largely maintaining the cellular structure of the wet foam.

The wet foam obtained in step (c) may be formed into a desired form before it is dried according to step (d) of the method. For example, the wet foam may be cast into a layer or sheet, or molded into a more detailed form before it is dried.

The drying of the wet foam in step (d) of the method of the present invention may be performed at a temperature of 5-95° C., 5-80° C., 10-70° C., 10-60° C., 10-50° C., 20-50° C., or 35-45° C.; or by subjecting the wet foam to a temperature of 5-95° C., 5-80° C., 10-70° C., 10-60° C., 10-50° C., 20-50° C., or 35-45° C.; until it reaches a liquid content of less than 98 wt %, or less than 90 wt %, less than 80 wt %, less than 70 wt %, less than 60 wt %, or even less than 50 wt % of the total weight of the wet foam. The drying is preferably performed in room temperature, but can also be performed in an oven, such as a convection oven or a microwave oven or by IR-radiation or any combination of these. The liquid content of the cellular solid material after drying may be 0 wt %, at least 1 wt %, at least 5 wt %, at least 10 wt %, at least 20 wt %, at least 30 wt %, or at least 40 wt %. The drying of the foam in step (d) may be performed at a pressure of 5-1000 kPa, 10-500 kPa, 20-400 kPa, 30-300 kPa, 40-200 kPa or preferably 50-150 kPa. Thus, resource intensive methods for drying the wet foam comprising an active substance, such as freeze-drying or supercritical drying, can be avoided and a cellular structure with closed cells may be obtained. The method according to the present thus provides for the preparation of a cellular material comprising closed cells. Drying performed at the temperatures and pressures according to the present invention has the advantage that the cellular solid material is less prone to cracking, especially when large components and sheets are formed. The porous structure may thus be maintained also when the foam has been dried.

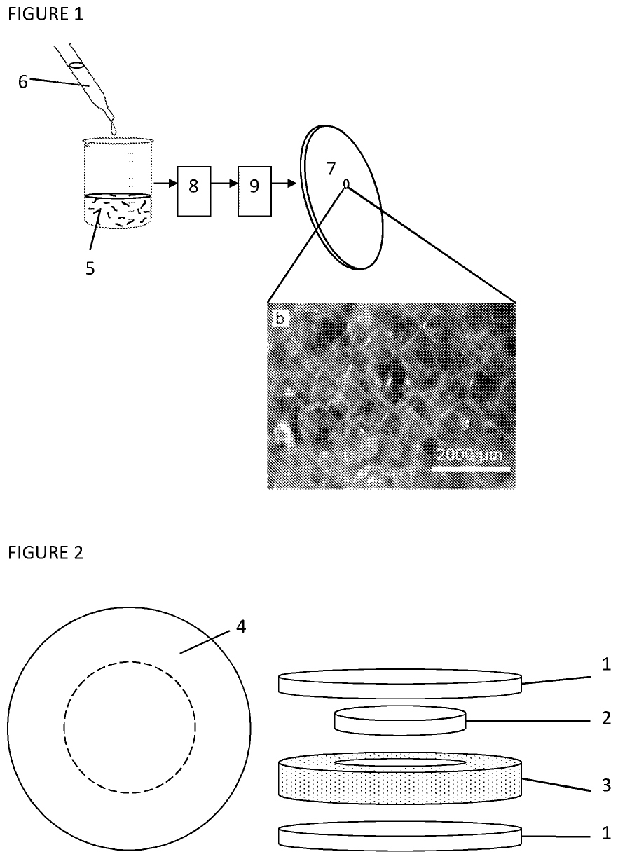

One embodiment of the method for preparing the structures according to the present invention is schematically illustrated in FIG. 1, where a cellular solid material comprising at least one active substance (7) is prepared by adding at least one active substance (6), optionally dissolved in a solvent, to a dispersion comprising cellulose nanofibers (5) followed by foaming (8) and casting (9) to a desired form that is subsequently dried.

The cellular solid material according to the present invention may be provided in a thickness of at least 0.05 mm, at least 0.1 mm, at least 0.5 mm, or at least 1 mm. The cellular solid material may be provided in a thickness up to and including 500 cm, 100 cm, or up to and including 50 cm.

By altering the processing conditions and the dispersion media during manufacturing and utilizing the inherent chemico-physical properties of CNF and molecular affinity between CNF and the active substance the hierarchical structure may be modified. This enables the preparation of formulations with tailored release properties of the active substance, from fast release to sustained release.

The structure for controlled release according to the present invention may be produced by the method according to the present invention. Alternatively, the structure for controlled release may be made by providing a wet foam comprising cellulose nanofibers (CNF) and a surfactant, to which the pharmaceutically active substance is added, and drying the wet foam to obtain a cellular solid material having a density of less than 1000 kg/m³, or less than 500 kg/m³.

The structure according to the present invention may be used in pharmaceutical compositions; medical devices, cosmetics; personal care; household applications; food science applications; veterinary medicinal compositions; industrial applications or in agriculture. Use of a cellular solid material comprising closed cells of cellulose nanofibers (CNF) and at least one active substance in a composition for controlled release of active substances is also an aspect of the present invention. Preferably, more than 10%, more than 50%, or more than 90% of the total volume of the cells of the cellular solid material are closed cells. The cellular solid material may be used as an excipient for an active substance, or a coating of at least one active substance, or a combination of these, for controlled release of said active substance.

A further aspect of the present invention is the use of a structure according to the present invention in applications selected from pharmaceutical compositions; medical devices cosmetics; personal care; household applications; food science applications; veterinary medicine; industrial applications and agriculture.

An additional aspect of the present invention is the use of a structure according to the present invention in therapy. The present invention also relates to the use of a cellular solid material comprising closed cells of cellulose nanofibers (CNF) and a pharmaceutical agent in a drug delivery composition for controlled release.

The invention will now be described by the following examples which do not limit the invention in any respect. All cited documents and references are incorporated by reference

### EXAMPLES

### Materials

Furosemide (crystal form I) and pepsin from porcine gastric mucosa was purchased from Sigma Aldrich. Commercial tablets of Furosemid-ratiopharm® (20 mg furosemide, Ratiopharm GmbH, Ulm, Germany) were purchased from a local pharmacy. Riboflavin was purchased from Unikem (Copenhagen Denmark). Lauric acid sodium salt was obtained from Acros Organics. Commercial tablets Vitamin B₂ 10 mg JENAPHARM® (10 mg riboflavin, mibe GmbH, Brehna, Germany) was purchased from a local pharmacy. Indomethacin (γ-form) and Glycidyltrimethylammonium chloride was purchased from Hawkins Pharmaceutics group and Sigma Aldrich, respectively. FaSSIF, FeSSIF & FaSSGF Powder was purchased from Biorelevant and used in the preparation of the FaSSGF media (pH 1.6, sodium taurocholate: 0.08 mM, lecithin: 0.02 mM, sodium chloride: 34.2 mM and hydrochloric acid: 25.1 mM, as specified by the producer Biorelevant) to this media 450 U mL⁻¹ of pepsin was added. In all examples, bleached sulfite pulp from spruce (never-dried pulp) was used in the production of the cationic nanocellulose (Nordic Paper Seffle AB, Sweden). The production of cationic nanocellulose is described in detail in literature (e.g. C. Aulin, et al., Biomacromolecules 2010, 11, 872-882) The pulp dispersion (in MilliQ water, dry content 16 wt %) was then diluted with isopropanol, 17 mL of isopropanol per g fibre (dry) and to this 0.08 g of NaOH per g fibre (dry) was added. The NaOH was dissolved in equal weight of MilliQ-water prior to addition. Cationic NFC was prepared by reacting pulp fibres and glycidyltrimethylammonium chloride in a 1:1 weight ratio. The reaction proceeded at 50° C. for 2 hours. The modified pulp was washed with an excess of MilliQ water and a suspension (ca. 2 wt % solid content) was homogenized using a high-pressure homogenizer (M-110P, Microfludics, U.S.) at 1650 bar (chambers 400/100 μm). A total of two passes were carried out. The amount of cationic groups was 0.44 mmol g⁻¹ fibre, attained by conductometric titration of chloride ions as described previously (Hasani, M; et al., Cationic surface functionalization of cellulose nanocrystals. Soft Matter 2008, 4 (11), 2238-2244). The CNF with 0.44 mmol of cationic groups g⁻¹ fibre was used in EXAMPLE 3. Cationic NFC with 0.13 mmol of cationic groups g⁻¹ fibre was prepared as described above but with the modification that the reaction temperature was gradually increased from 40 to 50° C. during one hour and then maintained at 50° C. for 1 h. Also, the chemically modified pulp-fibre (solid content 1.3 wt % in Milli-Q water) was high-pressure homogenized three times. The CNF with 0.13 mmol of cationic groups g⁻¹ fibre was used in EXAMPLES 1, 2, and 4. The nanofiber width was 5±1 nm and the fibre length was up to several μm, assessed by AFM height measurements. A fraction of non-fibrillated fibers could also be spotted in the final product, in particular in the CNF with the low cationic content.

### Example 1

### Furosemide

### Methods

### Preparation of Cellular Solid Materials Based on CNF and Furosemide.

Cellular solid materials comprising furosemide (7) were prepared by adding furosemide dissolved in 96 vol % EtOH (6) (concentration; 15.9 mg/mL and 58.6 mg/mL to prepare cellular solid materials with 21 wt % and 50 wt % furosemide, respectively) to a 0.28 wt % cationic CNF suspension (5) (pH=9.6) under vigorous magnetic stirring, see the schematic illustration in FIG. 1. A 0.28 wt % CNF suspension was prepared by diluting a stock suspension (1.321 wt % solid content) with Milli-Q water, adjusting the pH to 9.6 with 1M NaOH, followed by sonication (3 min, 90% amplitude, ½″ tip). The CNF/furosemide suspension was foamed (8) via an ultra-sonication step (80% amplitude, ½″ tip, 20 s sonication, 10 pause, Sonics Sonifier, 750 W) for 2 min. The foamed suspensions (20 g) were cast (9) (Petri-dished 8.8. cm in diameter) and dried in the dark at ambient conditions. The thickness of the cellular solid materials (n>12) were analyzed with light microscopy. The porosity was calculated from equation (1), using the theoretical density of the cellwall (ρ_{cellwall}) in the calculations:

ρ_{cellwall}=γ_{CNF}σ_{CNF}+ν_{active sub}ρ_{active sub} [2]

where ν_{CNF} is the volume fraction of the CNF and γ_{active sub} (=−ν_{CNF}) is the volume fraction of the active substance (furosemide). The densities ρ_{CNF}=1.5 g cm⁻³ and ρ_{active sub}=1.6 g cm⁻³ for CNF and furosemide, respectively, were used in the calculations of ρ_{cell wall}.

### Characterization

Scanning electron microscopy (SEM) images were obtained using a FEI Quanta 3D FEG (FEI, Oregon, USA). The cross-sections were obtained by cutting cellular solid materials with a sharp razor-blade. Samples were sputter-coated with 4 nm of gold.

Infrared spectroscopy (IR) spectra were acquired using an ABB MB3000 (ABB, Switzerland) in the total reflectance mode (attenuated total reflectance accessory) using 64 scans, with a resolution of 2 cm⁻¹. Measurements were performed on samples that had been dried overnight at 50° C. in vacuum oven.

A dissolution experiment was performed with Furosemid-ratiopharm® tablets (20 mg furosemide) and cellular solid samples containing ca. 7.3 mg of furosemide. The samples were about half the size a petri-dish (ca. 28 cm²) or ca. ⅛ of a petri-dish (ca. 6.6 cm²) for the cellular solid material loaded with 21 wt % and 50 wt % furosemide (dry weight basis), respectively. The experiment was conducted in a USP Apparatus 2 dissolution tester (Erweka, Heusenstamm, Germany) comprising beakers, where each beaker was provided with a stirring paddle and placed in a heated water bath. FaSSGF media (pH 1.6) was added to the beakers, the media contained pepsin (450 U mL⁻¹, porcine gastric mucosa Sigma Aldrich) and simulated gastric fluid. The composition of the media is given under “Materials”. The volume of FaSSGF media was 900 mL for the Furosemid-ratiopharm® tablets and 320 mL for the furosemide cellular solid materials. The experiment was conducted at 37° C., paddle stirring rate of 100 rpm for cellular solid samples (50 rpm for tablets). The cellular solid materials were floating on the FaSSGF media throughout the experiment, whereas the tablets disintegrated within a couple of minutes after addition to the media. One tablet or piece cellular solid materials was tested per beaker. Samples were withdrawn (2 mL and 5 mL for cellular solid materials and tablets, respectively) at 2, 5, 10, 20, 30, 60, 120, 240, 480 and 1440 min and were analyzed with UV-vis spectrophotometry (Agilent Cary 60 UV-vis) at a wavelength of 274 nm. The withdrawn samples were immediately replaced with equal amounts of new FaSSGF media containing 450 U mL⁻¹ of pepsin. The cumulative drug release in % was plotted as a function of time and all reported data-points were an average of three measurements.

### Results

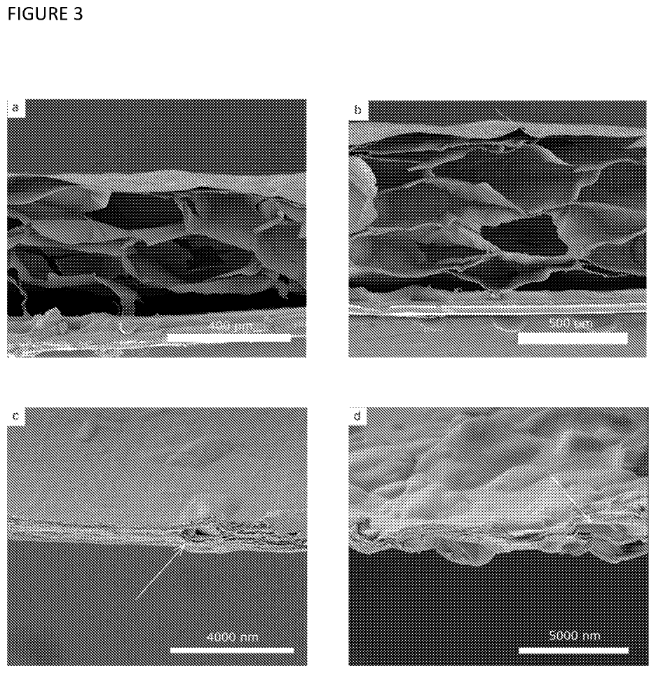

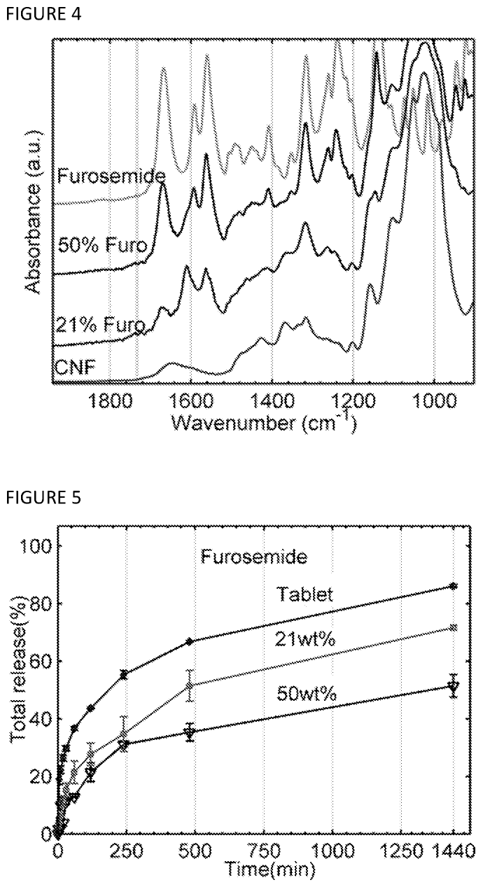

Cross-sections showing the cellular structure of the resulting cellular solid materials loaded with 21 wt % and 50 wt % furosemide, are presented in FIGS. 3a and 3b, respectively. Close-ups of the cell wall of the different cellular solid materials are presented in FIG. 3c (21 wt %) and FIG. 3d (50 wt %). There were undissolved furosemide particles present in the cell wall (arrows in FIGS. 3c and 3d), and many more particles could be observed in the cellular solid material loaded with more drug (50 wt %). These are bulk furosemide crystalline particles (form I) due to incomplete dissolution of the furosemide powder. The densities of the resulting cellular solid materials were 0.04 g cm⁻³ (porosity ϕ=97.5%) and 0.03 g cm⁻³ (ϕ=98.2%) for the furosemide/CNF cellular solid material loaded with 21 wt % and 50 wt % furosemide, respectively. IR data showed that the furosemide is largely present as an amorphous sodium furosemide salt in the cellular solid sample containing 21 wt % furosemide. This could be observed as a new band at 1608 cm⁻¹ (asymmetric COO— and C═O) and the reduction of the band height at 1670 cm⁻¹ (carboxylic acid dimer, COOH groups), in IR-spectra for the furosemide cellular solid material with 21 wt % furosemide in FIG. 4. The presence of amorphous furosemide salt was also evident in the sample with 50 wt % furosemide (as a shoulder at 1608 cm⁻¹) (L. H. Nielsen, et al., European Journal of Pharmaceutics and Biopharmaceutics, 85 (2013) 942-951). However, in this case the presence of crystalline furosemide (form I) was also apparent, compare with the spectra for the bulk furosemide. The resulting cellular solid materials exhibited positive buoyancy. The buoyant property of the cellular solid materials was yet an additional confirmation of the presence of mostly closed cells in the resulting cellular solid materials. Pieces of the cellular solid materials could be folded into various shapes, and two pieces of the cellular solid material containing 50 wt % furosemide were rolled and loaded into a hydroxypropyl methylcellulose capsule. The total furosemide content was 19.4 mg of furosemide, i.e. similar to a commercial furosemide tablet (20 mg). Upon wetting, the capsule swelled and the capsule wall dissolved, releasing the pieces. Upon release from the capsules, the cellular solid materials remained floating on the dissolution vessel. The release profile of the 21 wt % furosemide cellular solid material is slightly below that of the commercial tablet, see FIG. 5. At 50 wt % furosemide loading an even slower release is observed. All cellular solid materials were floating during the whole experiment for 24 h (the experimental measurement time) whereas the commercial tablets disintegrated within a couple of minutes.

### Example 2

### Riboflavin

### Methods Preparation of Cellular Solid Materials Based on Lauric Acid Sodium Salt and Riboflavin.

A 0.28 wt % CNF suspension was prepared by diluting a stock suspension (1.321 wt % solid content) with Milli-Q water, followed by sonication (3 min, 90% amplitude, ½″ tip) and subsequent adjustment of pH (^{˜}9.7, adjusted with 1M NaOH). The cellular solid materials were prepared by adding 0.395 mL dissolved lauric acid sodium salt in (96 vol %) EtOH (concentration 10 mg lauric acid per mL EtOH, and with 60 μl of 1M NaOH per mL EtOH) to 128 g of cationic CNF suspension (solid content 0.28 wt %, pH=9.7) under magnetic stirring. The bubbles were formed using an ultra-sonication step (80% amplitude, ½″ tip, 20 s sonication, 10 pause, Sonics Sonifier, 750 W) for 2 min. Riboflavin dispersed in water (solid content of 1 wt % or 6 wt % to prepare cellular solid materials containing 14 wt % or 50 wt % riboflavin (dry weight basis), respectively) was added to the wet foam under magnetic stirring. The wet foam (22 g) was cast in Petri-dishes (diameter: 8.8 cm) and dried at ambient conditions in the dark. The thin cellular solid materials were prepared in one step, but the thick cellular solid material was prepared by laminating several thin cellular solid materials pieces with wet foam (ca 15 g) in between the thin cellular solid material pieces and drying in petri-dishes (diameter: 8.8 cm) at ambient conditions in the dark. 206 g of suspension was used in total to create one thick cellular solid material sample containing 14 wt % riboflavin. The thickness of the thin cellular solid materials was analyzed with light microscopy (n>20) and the thickness of the thicker cellular solid materials was analyzed using a digital caliper. The porosity was calculated as described earlier using equations (1) and (2). The following densities were used in the calculations of the theoretical cell wall density; 1.65 g cm⁻³ (riboflavin), 1.5 g cm⁻³ (CNF). Lauric acid sodium salt was not considered because it was so low in content.

### Preparation of CNF Films (for Comparison)

CNF films containing 14 wt % riboflavin were prepared similar to that of cellular solid materials, however, after the sonication step of the CNF/lauric acid/EtOH suspension, the suspension was degassed to remove the air-bubbles and the riboflavin dispersion was added under slow magnetic stirring. The suspension (22 g) was cast in Petri-dishes (diameter: 8.8 cm) and dried under ambient conditions in the dark and stored in a desiccator with drying salt. A neat CNF film (reference film) was prepared by casting the neat CNF suspension and drying at ambient conditions. The lauric acid/CNF film used in the diffusivity experiments was prepared by laminating two dry lauric acid/CNF films (each prepared from 51 g of suspension) with degassed CNF/lauric acid/EtOH suspension, a total of 182 g degassed suspension was used in the preparation of one film. The thickness of the riboflavin loaded film was measured from Scanning electron microscopy images (n>60). The wet and dry thickness of the lauric acid/CNF film was analyzed (n>5) with Digimatic Indicator (Mitutoyo, USA).

### Characterization

Scanning electron microscopy (SEM) images were obtained using a FEI Quanta 3D FEG (FEI, Oregon, USA). The cross-sections were prepared by cutting cellular solid material samples with a sharp razor-blade and the films were torn. Samples were sputter-coated with 2 nm of Au prior to imaging.

X-ray diffraction (XRD) was performed using a PANalytical X'Pert PRO X-ray diffractometer (PW3040/60, PANalytical, Almelo, The Netherlands) with Cu Kα radiation (λ=1.54 Å, voltage 45 kV, current 40 mA) operated in reflection mode. Measurements were performed from 5 to 35° (2θ) using a step size of 0.0262606° (2θ).

### Diffusion Coefficient Measurements

Experiments were performed on the lauric acid/CNF film (diameter: 2.8 cm) using Franz diffusion cells (diffusion area A=2.01 cm²) consisting of a donor chamber (^{˜}4.2 mL, closed chamber with injection port) and heated receptor chamber (6.9 mL, magnetic stirring, one sampling port) that were placed into a magnetic stirrer block. The samples were clamped in-between the donor and receptor chamber. Experiments were performed at 37° C. Riboflavin in FaSSGF with 450 U mL⁻¹ pepsin was added to the donor side (4 mL, 8.3 mg riboflavin L⁻¹) and the receptor side was filled with media devoid of riboflavin. Samples (350 μL) were withdrawn from the receptor side at predetermined times and immediately replaced with equal amounts of new media. The amount of riboflavin was analyzed with fluorescence spectroscopy, FLOUStar OPTIMA MicroPlate Reader (BMG Labtech GmbH, Germany), using an excitation wavelength of λ_{exc}=450 nm and detection wavelength of λₑₘ=520 nm (front-face measurements). The total amount of riboflavin that had passed the film and the concentration difference, ΔC, on both sides of the film was calculated as a function of time. The diffusion coefficient, D, was calculated from the slope, s, of the steady state part (at short times 25-45 min, sink conditions, ΔC^{˜} constant) of the cumulative drug versus time plot. The slope divided by the diffusion area is the flux, F=s/A. The wet thickness (I=570±30 μm) of the film were used in the calculations of D (Crank, J., The mathematics of diffusion. 2d ed.; Clarendon Press: Oxford, Eng, 1975):

$$D = \frac{F * l}{Δ C} \qquad [ 4 ]$$

The reported diffusion coefficient for the lauric acid/CNF film is an average of two measurements. The dry thickness of the film was 89±14 μm.

### Dissolution Experiments

Experiments were performed to measure the cumulative drug release in % as a function of time for commercial riboflavin tablets and pieces of CNF cellular solid material/film. Experiments were performed on cellular solid material or film samples that contained ca. 2.3 mg of riboflavin. The size of the film (thickness 9 μm) and the thin cellular solid material (0.6 mm), both with 14 wt % riboflavin, was the same area (^{˜}¼ of a petridish, ca. 14 cm²) but had different thicknesses. The thick cellular solid material pieces were ca. 8×8×16 mm (H×W×L) and the thin cellular solid material sample loaded with 50 wt % riboflavin (0.7 mm thickness) had a top area of ca. 2.25 cm² (^{˜} 1/25 of a petridish). The experiment was conducted in a USP Apparatus 2 dissolution tester (Erweka, Heusenstamm, Germany), and a scaled down version of USP Apparatus 2 with special inserts and 250-mL vessels (Erweka DT 70, Heusenstamm, Germany). FaSSGF media (pH 1.6) containing pepsin (450 U mL⁻¹, porcine gastric mucosa Sigma Aldrich) was added to the beakers and simulated gastric fluid. Volume of FaSSGF media was 900 mL (normal USP Apparatus 2) for the tablets (JENAPHARM®) and 225 mL (scaled down USP Apparatus 2) for the riboflavin cellular solid materials or films. The experiments were conducted at 37±0.1° C. and pH 1.6, paddle stirring rate of 100 rpm (50 rpm for tablets). Dissolution experiments were conducted in two ways, either the cellular solid materials were floating (tablets and films did not float) or the samples were present in metal baskets residing on the bottom of the dissolution beakers. The experiments thus simulate two potential scenarios: one where the stomach has an upper gas-filled part or another when the stomach is completely filled with fluid and samples completely submerged in media. Samples (2 mL and 5 mL for cellular solid material/film and tablets, respectively) were removed at 2, 5, 10, 20, 30, 60, 120, 240, 480 and 1440 min and replaced with equal amounts of new FaSSGF media containing 450 U mL⁻¹ of pepsin. The amount of dissolved riboflavin was analyzed with UV-vis spectrophotometery (Agilent Cary 60 UV-vis) at a wavelength of 266 nm. All reported values are an average of three measurements.

### Results

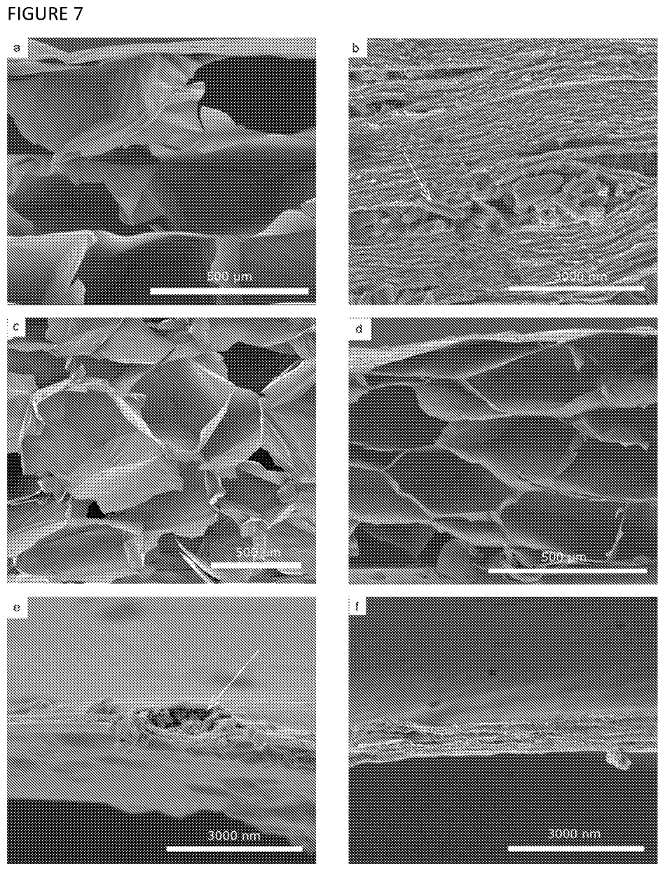

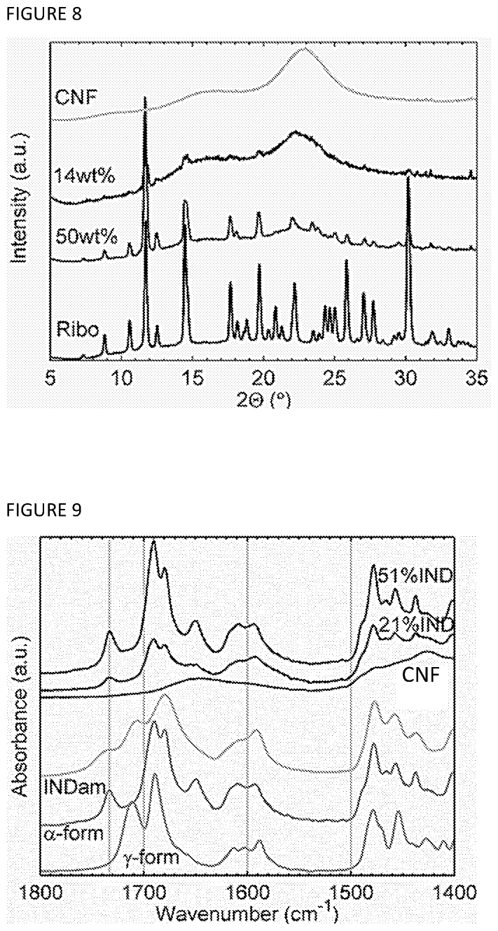

Several different examples of resulting CNF based cellular solid materials were prepared to demonstrate the versatility of this preparation route. Cellular solid materials of different thicknesses, shapes and drug loading (up to 50 wt %) was prepared, which is illustrated in FIGS. 6 a-e. The flexibility of a thin cellular solid material (7) (FIG. 6a) allowed the material to be folded or rolled (6b) into smaller objects and delivered in a capsule (13) suitable for swallowing (FIG. 6c). Also a ring structure of CNF was made (FIG. 6d), as well as pieces of cellular solid materials of different thickness (FIGS. 6a and 6e). The cellular solid materials had a closed cell structure. SEM micrographs are presented in FIG. 7 showing the structure neat CNF/lauric acid cellular solid material (a), riboflavin-loaded film (14 wt %, b) and CNF/lauric acid cellular solid materials loaded with 14 (c) and 50 wt % (d) riboflavin. The structure shown in FIG. 7c is that of the thicker CNF cellular solid material with 14 wt % riboflavin. A close-up of the cell wall is given in FIG. 7e of the cellular solid material loaded with 50 wt % riboflavin, displaying riboflavin crystals (arrow) that are embedded in the CNF based cell wall. Riboflavin crystals (arrow) can also be observed in the film (b). As a comparison the cell wall of a neat CNF/lauric acid cellular solid material is given in FIG. 7f. The density of the resulting cellular solid materials measured: 0.01 g cm⁻³ (Lauric acid/CNF, ϕ=99.0), 0.02 g cm⁻³ (14 wt % riboflavin, ϕ=98.7), 0.03 g cm⁻³ (50 wt % riboflavin, ϕ=98.0). An increase in density (and decrease in porosity) was observed when the cellular solids materials were loaded with more riboflavin. The riboflavin used in the present study displayed XRD peak typical for anhydrate I form (e.g. compared with Figure A in WO2005/014594). For the cellular solid material and the film with 14 wt % or 50 wt % riboflavin loading, no changes to the crystal form of the riboflavin could be observed when compared to that of the neat riboflavin powder sample, see XRD diffractogram in FIG. 8.

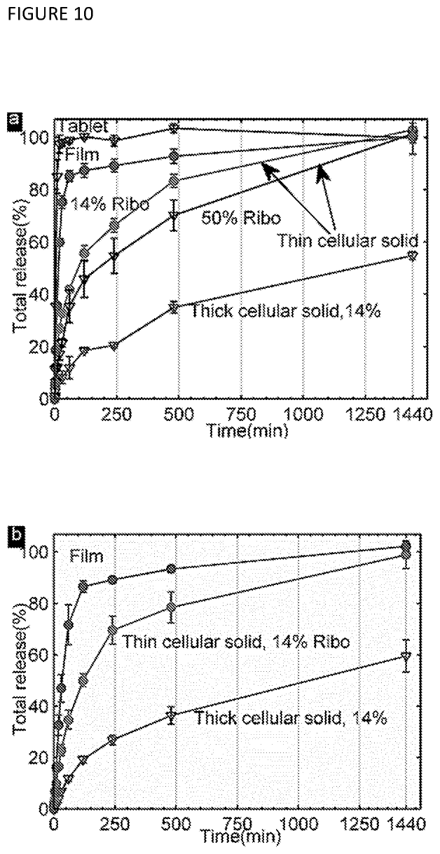

The kinetics of drug release was evaluated in simulated FaSSGF media (pH 1.6) containing 450 U mL⁻¹ pepsin. All riboflavin loaded CNF samples contained the same amount (ca. 2.3 mg) of riboflavin. The cellular solid materials remained buoyant throughout the drug release studies, which suggests a closed-cell cellular solid material structure with the air-bubbles remaining intact during the experimental time frame (only 24 hour were tested). The closed-cell structure is in line with previous SEM images. The entrapped air-bubbles and the high porosity provide the cellular solid materials with buoyance power. The release characteristics from cellular solid materials with different thicknesses and riboflavin loading are presented in FIG. 10. As a comparison, commercial tablets and neat CNF films with 14 wt % riboflavin were also included. Experiments were conducted in two ways; samples moving freely in the dissolution media (FIG. 10a) or samples in metal baskets residing on the bottom of the dissolution beakers (results presented in FIG. 10b). The drug release data for CNF based samples was similar in both cases, compare FIGS. 10a and 10b. The initial drug release rate was quite fast for all CNF based samples and due to riboflavin located at the outer surface of the samples facing the dissolution media (the samples were not washed prior to testing), see FIGS. 10a and 10b. The release profile from the commercial tablet (Vitamin B2 10 mg JENAPHARM®) was slightly quicker than the film, due to a combination of fast disintegration of the tablet (within a couple of minutes) and probably another crystal form of the riboflavin (which could not be identified), see FIG. 10a. As expected, the CNF based film (thickness 9 μm) containing 14 wt % riboflavin, released all drug rapidly, whereas the release profile for the cellular solid materials also highly depended on the thickness. The diffusion coefficient for riboflavin through a neat CNF/lauric acid film is quite high in the wet state, i.e. 2.2×10⁻⁶ (±0.13 10⁻⁶) cm² s⁻¹, which is approximately one order of magnitude lower compared to small molecule diffusion in water (around 10⁻⁵ cm² s⁻¹ at 20° C.) (Freitas, R. A., Nanomedicine, Landes Bioscience: Austin, Tex., 1999). In other words, the riboflavin diffusivity should have little influence on the overall drug release kinetics of the films presented in FIG. 10. The observed fast drug release from the CNF based films herein fit well with that assumption. The exact diffusivity in CNF films will depend on factors such as nanofiber packing, which is a consequence of e.g. the degree of nanofibrillation and surface modification of the nanofibers. The present lauric acid/CNF film contained some larger non-fibrillated fragments, observed with the naked eye.

Both the cellular solid material loaded with 14 wt % (thickness 0.6 mm) and the one with 50 wt % riboflavin (0.7 mm) were of comparable thickness and the drug release profiles overlapped, see FIG. 10a. The cellular solid materials presented a slower release compared to the film. The film samples can be regarded as a collapsed thin cellular solid material sample and they were prepared from the same type of suspension, see experimental section for details. To further illustrate the effect of structure on the drug release properties three different CNF based samples types were explicitly compared in FIG. 10b. The samples contained the same amount of riboflavin and total solid content (riboflavin+CNF+lauric acid) but have different hierarchical structures (film or cellular solid material) and sample dimensions. The top surface area (also bottom) of the film (thickness 9 μm) and the thin cellular solid material (0.6 mm thick) were comparable, however the thickness is different due to the presence of air-bubbles. The thicker cellular solid material piece had the dimensions 8×8×16 mm (H×W×L) and thus a much smaller total surface area. As expected, the thicker cellular solid material piece had the slowest drug release profile whereas the film released the fastest. These results clearly illustrate how the inherent properties of CNF can be exploited to easily alter the structure of CNF based drug-delivery systems in order to precisely modify the drug release profile.

### Example 3

### Indomethacin

### Methods

### Preparation of Cellular Solid Material, Amorphous Indomethacin and α-Form of Indomethacin

Cellular solid materials (7) loaded with indomethacin were prepared using a simple solvent-casting step, schematically illustrated in FIG. 1. The cationic CNF (5) was diluted with MilliQ-water to 0.28 wt % and dispersed by ultrasonication at 70% amplitude for 120 s (Sonics Sonifier, 750 W, ½″ tip, 20 s pulse, 10 s pause). The pH of the CNF suspension was 7.8. Indomethacin was dissolved in 96 vol % ethanol (concentration 15 mg indomethacin per mL of EtOH) (6) and added drop-wise to the aqueous cationic CNF suspension under vigorous magnetic stirring. The resulting suspension was ultrasonicated (80% amplitude for 120 s, 20 s pulse, 10 s pause, water cooling). The foaming properties of the suspension were highly dependent on the pH of the suspension and a wet-stable foam was obtained in the case of 21 wt % (dry weight content) indomethacin loading. The pH of the resulting suspension used in the preparation of 21 wt % cellular solid material was 4.9. To create the cellular solid material with 21 wt % indomethacin, the wet foam (22.8 g) was casted (9) into Petri-dish (8.8 cm) and dried at ambient conditions for two days. The cellular solid materials adhered strongly to the bottom of the Petri-dishes. The thickness of typical samples used in dissolution testing was 1180±260 μm for the cellular solid material (measured with a caliper and light microscopy). The density of the cellular solid material was 0.01 g cm⁻³ (porosity, ϕ=99.2%, calculated as described before using equations 1 and 2, and a density for indomethacin of ρ_{indo}=1.379 g cm⁻³).

### Preparation of Films (as a Comparison)

Films were made using the same protocol as in the case of foams, but introducing a degassing step after the sonication step to remove air-bubbles. The pH of the resulting suspension used in the preparation of the 51 wt % film was pH=5.5. To form coherent films, the suspension was degassed under reduced pressure to remove air, solvent-cast (28.8 g and 18.6 g of suspension for films with 21 and 51 wt % indomethacin, respectively) in Petri-dishes (8.8 cm) and dried in a heating cabinet at 52° C. for two days. Neat CNF films were prepared by casting the neat cationic CNF suspension, followed by drying at 52° C.

The films adhered strongly to the bottom of the Petri-dishes. The resulting films were 19±1.6 μm and 15±2.7 μm (Digimatic Indicator, Mitutoyo, USA) at 21 wt % and 51 wt % indomethacin loading, respectively. The α-form of indomethacin was prepared by adding distilled water to dissolved indomethacin in ethanol (approx. 80° C.). The precipitate was filtered and dried under vacuum for 24 h at room temperature. Amorphous indomethacin was prepared by melting indomethacin (γ-form) on a hot plate at 170° C. followed by quench cooling on a cold (room temperature) metal plate.

### Characterization

Light microscopy images of the cellular solid material were acquired using a Zeiss light microscope (stereo Discovery V.8, Zeiss, Germany) and the AxioVixion Rel 4.8 software. The diameter of the cells in the cellular solid material was analyzed from n=75 cells.

IR spectra were obtained using an ABB MB3000 (ABB, Switzerland) in the total reflectance mode (attenuated total reflectance accessory). Measurements were performed of dry samples and spectra were collected from 400-4000 cm⁻¹ (64 scans, with a resolution of 2 cm⁻¹).

Dissolution experiments were conducted to determine the intrinsic dissolution curve in mg cm⁻² as a function of time (min) and the cumulative drug release curve in % as a function of time (min). The release in mg cm⁻² was derived as described previously using the theoretical surface area occupied by indomethacin in the calculations (K Löbmann, et al., Eur. J. Pharm. Biopharm. 2013, 85, 873-881). Studies were performed on films and cellular solid material in petri-dishes (surface area 58.6297 cm⁻²) using the fixed disk method (M G Issa, et al., Dissolut. Technol. 2011, 18, 6-13). As described above, the films and cellular solid materials adhered strongly to the surface of the petri dishes, only allowing drug release from the open side of the petri dishes. Magnets were fastened (with tape) under the petri dishes to avoid floating. Intrinsic dissolution of the crystalline and amorphous indomethacin was performed using the rotating-disk system (Wood's apparatus) (Issa, 2011). Powder compacts (150 mg) were obtained from direct compression into stainless steel cylinders (surface area 0.7854 cm⁻²) using a hydraulic press (Hydraulische Presse Model IXB-102-9, PerkinElmer, Germany) and a pressure of 124.9 MPa for 10 s. The samples were place in 900 mL of 0.01 M phosphate buffer (pH 7.2, 37° C.) using a rotation speed of 50 rpm (USP Apparatus 2 dissolution tester; Erweka, Heusenstamm, Germany). Samples (5 mL) were removed at predetermined times (5, 10, 20, 40, 120, 240, 1440 min) and immediately replaced with phosphate buffer. The amount of indomethacin was analysed using UV-vis spectrophotometry (Evolution 300, Thermo Fisher Scientific, USA), at A=263 nm. All values are an average of three measurements, except for the 24 h data point for the cellular solid material, which was a single measurement.

### Results

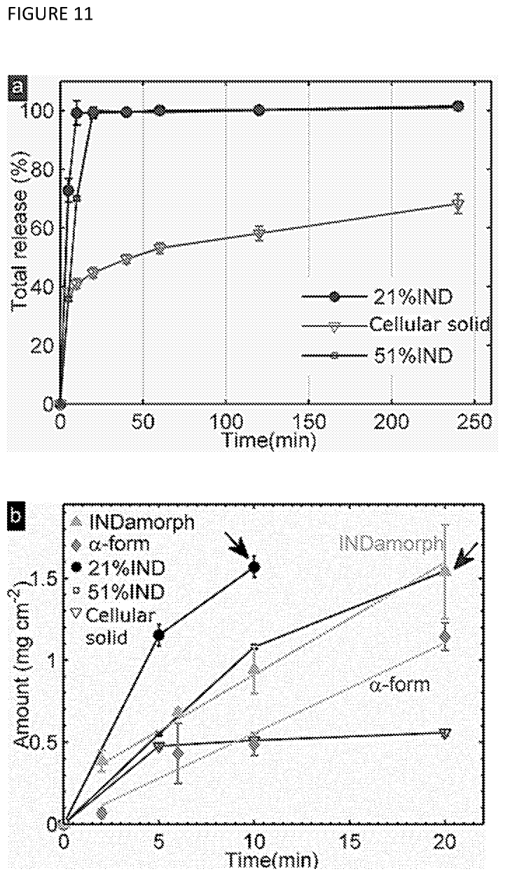

The cellular solid material, created by combining indomethacin and CNF, had a closed cell-structure with cells in the size of 540±180 μm, as observed with light microscopy. The resulting cellular solid material density was 0.01 g cm⁻³, which corresponds to a cellular solid material porosity of 99.2%. For the film loaded with 21 or 51 wt % indomethacin and the cellular solid material with 21 wt % indomethacin bands appeared at; 1733 cm⁻¹, 1690 cm⁻¹, 1679 cm⁻¹, 1650 cm⁻¹, see FIG. 9. The four bands are characteristic for crystalline indomethacin in the α-form, (S A Surwase, et al., Molecular Pharmaceutics 2013, 10, 4472-4480) thus the IR results suggested that the film and cellular solid material contained crystalline matter. The IR-spectrum of the cellular solid material is not included in FIG. 9, because it overlapped with that of the 21 wt % film. There was a difference in the region 1615 to 1590 cm⁻¹. These bands are due to the indol and chlorobenzyl ring deformation, as well as the ether C—O stretching (C J Strachan, T. Rades, K. C. Gordon, Journal of Pharmacy and Pharmacology 2007, 59, 261-269). The IR-spectra at 21 wt % indomethacin was more similar to that of the amorphous indomethacin in this region, indicating the presence of an amorphous fraction of the drug. In particular, the vibration at 1610 cm⁻¹ was less pronounced than that at 1592 cm⁻¹. At 51 wt % loading, however, such interpretations could not be made, because the more pronounced bands of α-indomethacin made detection difficult. As can be seen from the drug release profile in FIG. 11a, the drug was released very quickly for the films, with the complete drug being released after 10 and 20 min for the 21 wt % and 51 wt % drug-loaded films, respectively. Compared to the films, the cellular solid materials displayed a much slower release profile. The cumulative drug release was initially fast (see the total drug-release profile for the cellular solid materials in FIG. 11a), which was followed by a much slower release. After 24 h, 99.2% of the indomethacin had been released from the cellular solid material (last point not included in the figure). The porosity was ϕ=99.2%, and the cellular solid material was about 60 times thicker compared to films. The intrinsic dissolution curves for the drug-loaded films and cellular solid materials are depicted in FIG. 11b and were compared to the intrinsic dissolution for the pure crystalline α-indomethacin and amorphous indomethacin. The 21 wt % film showed a much faster release compared to the pure amorphous indomethacin; 2 times more drug per area was released from the film compared to the amorphous indomethacin after 5 minutes (and more than 4 times more drug per area compared to the α-polymorph). After 10 minutes all indomethacin present in this film was released (left arrow in FIG. 11b). On the other hand, the dissolution kinetics of the 51 wt % film was comparable to that of the amorphous indomethacin, which was still much faster compared to the α-polymorph. Theoretically, the dissolution for a mixture of alpha-indomethacin and amorphous drug is expected to lie in between the release profiles for the pure amorphous and the alpha-form of indomethacin. The dissolution of the 21 wt % cellular solid material showed an initial fast release, and then a much slower dissolution due to the diffusion controlled mechanism as described above.

### Example 4

### Riboflavin

### Methods

### Preparation Cellular Sold Material Coating of a Riboflavin Cellular Solid Material (“Ravioli”)

A 0.28 wt % CNF suspension was prepared by diluting a stock suspension (1.321 wt % solid content) with Milli-Q water, followed by sonication (3 min, 90% amplitude, ½″ tip) and subsequent adjustment of pH (^{˜}9.7). Cellular solid materials were prepared by adding 0.395 mL dissolved lauric acid sodium salt in 96 vol % EtOH (concentration 10 mg/mL EtOH, and with 60 μl of 1M NaOH per mL EtOH) to 128 g of cationic CNF suspension (solid content 0.28 wt %, pH ^{˜}9.7) under magnetic stirring. Bubbles were formed using an ultra-sonication step (80% amplitude, ½″ tip, 20 s sonication, 10 pause, Sonics Sonifier, 750 W) for 2 min. Riboflavin dispersed in water (solid content of 1 wt %) in order to prepare cellular solid materials containing 14 wt % riboflavin, was added wet foam under magnetic stirring. The wet foam (22 g) was cast in Petri-dishes (diameter: 8.8 cm) and dried at ambient conditions in the dark. The thin cellular solid materials were prepared in one step, but the thick cellular solid material was prepared by laminating several thin cellular solid material pieces with wet foam (ca 15 g) in between the thin cellular solid pieces and drying in petri-dishes (diameter: 8.8 cm) at ambient conditions in the dark. A thick lauric acid/CNF cellular solid material was prepared using a total of 97 g of suspension per sample. The final cellular solid materials were stored in a desiccator with drying salt. The lauric acid/CNF film was prepared similar to that of cellular solid materials, however, after the sonication step of the CNF/lauric acid/EtOH suspension, the suspension was degassed to remove the air-bubbles. Two dry lauric acid/CNF films (each prepared from 51 g of suspension) were laminated with CNF/lauric acid/EtOH suspension; a total of 182 g degassed suspension was used in the preparation of one film. To prepare a ravioli configuration (4), (illustrated in FIG. 2), the 14 wt % riboflavin cellular solid materials were cut out in a circular piece (35 mm in diameter) (2). Two lauric acid/CNF cellular solid pieces (1) (54.5 mm in diameter) were assembled as presented in FIG. 2, using 3.7 g of wet foam (3) that upon drying glued together the two cellular solid materials (1). The configuration was dried at room temperature.

### Characterization

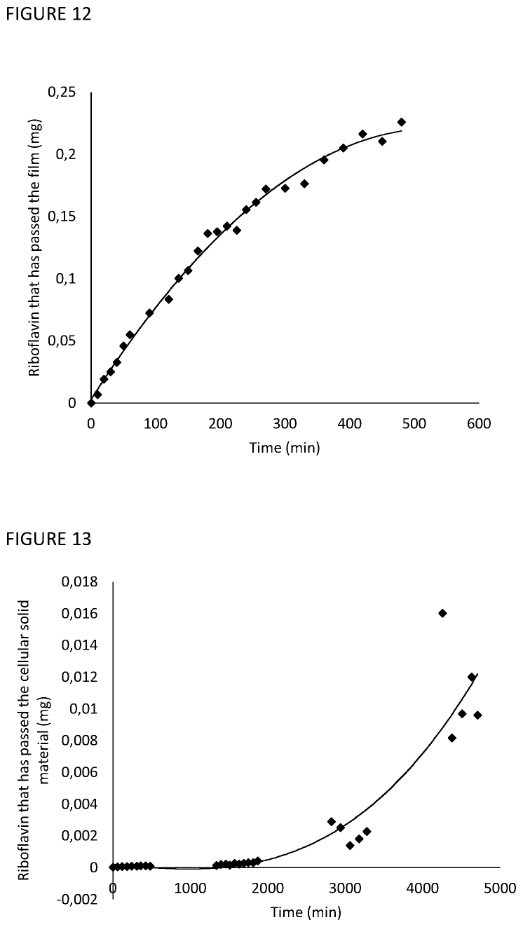

Diffusion coefficient measurements were performed on the lauric acid/CNF cellular solid material and the lauric acid/CNF film (diameter: 2.8 cm) using Franz diffusion cells (diffusion area A=2.01 cm²) consisting of a donor chamber (^{˜}4.2 mL, closed chamber with injection port) and heated receptor chamber (6.9 mL, magnetic stirring, one sampling port) that were placed into a magnetic stirrer block. Experiments were performed at 37° C. Riboflavin in a modified PBS buffer (8 g L⁻¹ NaCl, 2.38 g L⁻¹ Na₂HPO₄, 0.19 g L⁻¹ KH₂PO₄, pH adjusted to 7.5) was used and 3.8 mL of buffer containing 90 mg L⁻¹ riboflavin was added to the donor chamber (t=0). The receptor side was filled with media devoid of riboflavin. Samples (350 μL) were withdrawn from the receptor side at predetermined times and immediately replaced with equal amounts of new media. The amount of riboflavin was analyzed with fluorescence spectroscopy, FLOUStar OPTIMA MicroPlate Reader (BMG Labtech GmbH, Germany), using an excitation wavelength of λ_{exc}=450 nm and detection wavelength of λₑₘ=520 nm (front-face measurements). The total amount of riboflavin that had passed the film and the concentration difference, ΔC, on both sides of the film was calculated as a function of time. The diffusion coefficient, D, was calculated from the slope, s, of the steady state part (at short times 30-50 min, sink conditions, ΔC ^{˜} constant) of the cumulative drug versus time plot, see plot in FIG. 12. The slope divided by the diffusion area is the flux, F=s/A. The wet thickness of the film (l=460±12 μm) was used in the calculation of the diffusion coefficient D according to equation [3] (Crank, J., The mathematics of diffusion. 2d ed.; Clarendon Press: Oxford, Eng, 1975; p viii, 414 p). The reported diffusion coefficient for the lauric acid/CNF film is an average of two measurements. The diffusion coefficient for a lauric acid/CNF cellular solid material (density 0.01 g cm⁻³, wet-thickness ^{˜}5 mm) was estimated using the same Franz diffusion cell setup. Evaporation/leakage from the sides of the cellular solid material (when it was clamped in the Franz diffusion cell) was effectively minimized by covering the side of the cellular solid material with Vaseline (Vaseline pure, Democal AG, Bern) and then rapping with Parafilm®. Any observed evaporation from the receptor side was replaced with new buffer. 3.5 or 3.7 mL of riboflavin (90 mg L⁻¹) in modified PBS buffer (composition described above), was added to the donor chamber (t=0) and samples were withdrawn from the receptor chamber (350 μl) and replaced with fresh media at predetermined times. The samples were analyzed using a FLOUStar Omega MicroPlate Reader as described before. The diffusion coefficient was calculated from the time-lag method (Crank, J., The mathematics of diffusion. 2d ed.; Clarendon Press: Oxford, Eng, 1975; p viii, 414 p). It follows that the time-lag, 8, which is the time before the steady-state flow rate has been established, and the thickness of the sample, l, can be used to calculate the diffusion coefficient, D_{comp}:

$$θ = \frac{{l}^{2}}{6 {D}_{comp}} \qquad [ 5 ]$$

It was assumed that the diffusion coefficient is independent of the riboflavin concentration, i.e. D is a constant. This also means that the time-lag, 8, is independent of riboflavin concentration (from Equation 5).

### Results

It is possible to prepare a ravioli configuration (illustrated in FIG. 2) using a combination of cellular solid materials and wet foam. To understand the influence of a cellular solid material coating on the diffusivity, the diffusivity in a lauric acid/CNF cellular solid material versus a lauric acid/CNF film was compared. The riboflavin diffusivity in the lauric acid/CNF film was obtained from the steady state part of the total amount of riboflavin that had passed the film as a function of time, see FIG. 12 for a typical plot. The diffusion coefficient was Dₒ=4.7×10⁻⁶ (±0.7×10⁻⁶) cm² s⁻¹. A typical plot showing the total amount of riboflavin that has passed the cellular solid material as a function of time is presented in FIG. 13. For all cellular solid materials tested, approximately 2000 minutes (more than one day) was needed before the riboflavin started to be detectable on receptor side, demonstrating the significant reduction in diffusivity due to presence of air-bubbles in the cellular solid material. To find the time-lag, θ, a linear curve was fitted to the established “steady-state” part of the curve and the intercept with the time-axis gave θ. Using Equation (5) the diffusion coefficient could be obtained; D_{comp}=3×10⁻⁷ (±0.4×10⁻⁷) cm² s⁻¹. The calculated value is an average of two measurements. From the above results it is possible to calculate the ratio between the diffusivity in the film to that of the cellular solid material, i.e. Dₒ/D_{comp}=15.7, where Dₒ is the diffusivity in the film and D_{comp} is the diffusivity in the cellular solid material. In other words, the diffusivity decreased more than one order of magnitude for the cellular solid material when compared to a film.

1. A structure for the controlled release of at least one active substance, wherein the structure comprises at least said active substance and a cellular solid material comprising cellulose nanofibers (CNF), wherein the structure has a density of less than 1000 kg/m³, and more than 10% of the total volume of the cells of the cellular solid material are closed cells.

2. The structure according to claim 1, wherein more than 50% of the total volume of the cells of the cellular solid material are closed cells.

3. The structure according to claim 1 or 2, wherein the controlled release is a delayed release, sustained release, fast release or burst release.

4. The structure according to claim 3, wherein the controlled release is a sustained release.

5. The structure according to any one of claims 1-4, wherein the cellular solid material is used as an excipient for the active substance.

6. The structure according to any one of claims 1-5, wherein the cellular solid material is used as a coating.

7. The structure according to any one of claims 1-6, wherein the active substance is selected from pharmaceutically acceptable agents, catalysts, chemical reagents, nutrients, food ingredients, enzymes, bactericides, pesticides, fungicides, disinfectants, fragrances, flavours, fertilizers, and micronutrients.

8. The structure according to any one of claims 1-7, wherein the active substance is a pharmaceutically acceptable agent.

9. The structure according to claim 8, wherein the pharmaceutically acceptable agents is a therapeutically, prophylactically and diagnostically active substance.

10. The structure according to claim 9 for administration selected from any one of oral, topical, transdermal, subdermal, intracavitary, and intranasal administration of the pharmaceutically acceptable agent.

11. The structure according to any one of claims 1-10, which is in a form selected from any one of a tablet; a pill; a lozenge; a capsule; a granule; a sachet; a chewing gum; a layered structure; an injectable drug carrier; a gel; transdermal patches; a bioadhesive; a scaffold; a device; and an implant.

12. The structure according to any one of claims 1-11, which is a floating drug delivery structure.

13. The structure according to any one of claims 1-12, which is buccal mucosa drug delivery structure.

14. A method for preparing a structure according to any one of claims 1-13, comprising:
a) providing a dispersion comprising CNF in an aqueous solvent,
b) adding at least one active substance to the dispersion in (a) to obtain a mixture;
c) preparing a wet foam from the mixture obtained in (b), wherein the wet foam has a density less than 98% of the density of the mixture prior to foaming; and
d) drying the wet foam obtained in (c) to obtain a structure comprising a cellular solid material and at least one active substance

15. A method according to claim 14, wherein the active substance is a pharmaceutically acceptable agent.

16. Use of a cellular solid material comprising closed cells of cellulose nanofibers (CNF) and at least one active substance in a composition for controlled release of said active substance.

17. Use according to claim 16, wherein the cellular solid material comprises closed cells.

18. The structure according to any one of claims 1-13 for use in pharmaceutical compositions; medical devices, cosmetics; personal care; household applications; food science applications; veterinary medicinal compositions; industrial applications or in agriculture.

19. A structure according to any one of claims 1-13 for use in therapy.
